(19) United States
(12) Patent Application Publication
(10) Pub. No.: US 2006/0009679 A1
(43) Pub. Date: Jan. 12, 2006

(54) Electronic endoscope system capable of detecting inserted length

(75) Inventors: Eiichi Ito, Chiba-ken (JP); Mitsuhiro Matsumoto, Tokyo (JP); Koji Tsuda, Saitama-ken (JP); Kohei Iketani, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/175,175

(22) Filed: Jul. 7, 2005

(30) Foreign Application Priority Data
Jul. 8, 2004 (JP) 2004-202136

(51) Int. Cl. A61B 1/00

(52) U.S. Cl. 600/117

(57) ABSTRACT

An endoscope system includes an endoscope having an insertion section to be inserted into a body cavity, a plurality of sensors that detects environmental condition thereof, the plurality of sensors being arranged along a longitudinal axis of the insertion section, a processor that obtains information related to an inserted amount of the insertion section in accordance with detection results of the plurality of sensors, and a monitor device that displays information related to the inserted amount of the insertion section.

### BACKGROUND OF THE INVENTION

The present invention relates to an electronic endoscope system that is configured to detect a length of an inserted part of an endoscope.

Generally, when an inside tissues of a body cavity of a patient are observed, electronic endoscope is used to capture images. The electronic endoscope is generally provided with an image sensor such as a CCD (Charge Coupled Device), which is equipped at a distal end portion of a flexible tube. Cables for transmitting electrical signals and optical fibers for transmitting light to illuminate the object are accommodated inside the flexible tube. When such an endoscope is used, an operator is occasionally required to judge how far the flexible tube of the endoscope is inserted into the body cavity.

In order to determine the state of the endoscope that is inserted into a body cavity, there is known an endoscope system utilizing scale marks provided on the outside surface of the flexible tubes.

Judging the inserted length by the scale marked on the flexible tube of an endoscope may cause an inconvenience to an operator who is observing the images captured by the endoscope on a monitor screen. A skilled operator may estimate the inserted length based on a location of the distal end of the endoscope which is known by observing the images displayed on the monitor. However, the exact length cannot be known in such a method. Further, with only the scale, no information on the inserted length during the operation is recorded, i.e., when the video images recorded through the endoscope are viewed afterward the operation, no data to determine which part of a body is displayed on the screen is provided.

### SUMMARY OF THE INVENTION

The present invention is advantageous in that an improved endoscope system is provided, which system is capable of displaying information regarding the inserted length of the endoscope on a monitor.

According to an aspect of the invention, there is provided an endoscope system, which includes an endoscope having an insertion section to be inserted into a body cavity, a plurality of sensors that detects environmental condition thereof, the plurality of sensors being arranged along a longitudinal axis of the insertion section, a processor that obtains information related to an inserted amount of the insertion section in accordance with detection results of the plurality of sensors, and a monitor device that displays information related to the inserted amount of the insertion section.

Optionally, the processor may identify a sensor located at a boundary between an inserted part and non-inserted part of the insertion section in accordance with the detection results of the plurality of sensors.

In a particular case, the information related to the inserted amount of the insertion section obtained by the processor may be an actual length which is displayed on the monitor as a character string. Alternatively or optionally, the information related to the inserted amount of the insertion section obtained by the processor may be displayed on the monitor as an image indicating an inserted length.

Optionally, the image indicating the inserted length may include an insertion length indicator that shows the inserted amount of the insertion section with respect to an entire length of the insertion section.

Further, the insertion length indicator may be displayed on the monitor device together with a diagram of a human body.

The endoscope system may further include a storing system that stores the information related to the inserted length of the insertion section.

Optionally, the information related to the inserted length may be overlaid on an observation image on the monitor device.

Still optionally, the insertion section may be defined by a front end and a rear end, wherein the sensors are arranged between the front end and the rear end in a predetermined regularity.

In particular, the plurality of the sensors may be arranged such that every two adjoining sensors are spaced from each other in an axial direction of the insertion section, while the every two adjoining sensors are located at opposite positions in a circumferential direction of the insertion section.

Optionally, the inserted length may be determined by identifying a sensor which is closest to the front end of the insertion section among other sensors that are arranged on a part of the insertion section that is not inserted into the body.

The processor may be configured to determine a boundary between the inserted part and non-inserted part of the insertion section in accordance with a difference between detection results adjoining ones of the plurality of sensors.

Further, the processor may be configured to determine a boundary between the inserted part and non-inserted part of the insertion section in accordance with the individual detection results of the plurality of sensors.

The plurality of sensors may include at least one of temperature sensors, photo sensors, pressure sensors, vibration sensors, wetness sensors and humidity sensors.

In a particular case, the sensors may include the optical sensors. In such a case, and if the endoscope comprises a flexible tube, the sensors may be arranged on an inner surface of the flexible tube, and at least portions of the flexible tube facing the optical sensors may be formed of optically transmittable materials.

The plurality of sensors may include at least two types of sensors, each including the plurality of sensors. In such a case, the at least two types of sensors may include at least two of temperature sensors, photo sensors, pressure sensors, vibration sensors, wetness sensors and humidity sensors.

According to another aspect of the invention, there is provided an endoscope system, which includes an endoscope having an insertion section to be inserted into a body cavity for observation of an object therein, a plurality of sensors that detects environmental condition thereof, the plurality of sensors being arranged along a longitudinal axis of the insertion section, a monitor device that displays an image in accordance with input signals, an observation image generating system that captures an image of the object and outputs a signal representing the captured image to the monitor device, a controller that identifies a boundary of inserted part and non-inserted part of the insertion section in accordance with detection results of the plurality of sensors, the controller generates length information which is transmitted to the monitor device for display.

### BRIEF DESCRIPTION OF THE ACCOMPANYING DRAWINGS

FIG. 1 is a diagrammatic view of a configuration of an endoscope system according to an embodiment of the present invention;

FIG. 2 is a diagrammatic view of a configuration of an endoscope system, which is being inserted into a human body;

FIG. 3 is a perspective view of an insertion portion of a flexible tube to be inserted in a body cavity;

FIG. 4 is a cross-sectional view of the insertion portion of the flexible tube taken along line A-A shown in FIG. 3;

FIG. 5 is a cross-sectional view of a first modification of the insertion portion of the flexible tube taken along line A-A shown in FIG. 3.

FIG. 6A is a cross-sectional view of a second modification of the insertion portion of the flexible tube taken along line A-A shown in FIG. 3;

FIG. 6B is a cross-sectional view of a third modification of the insertion portion of the flexible tube taken along line A-A shown in FIG. 3;

FIG. 7 is a cross-sectional view of a fourth modification of the insertion portion of the flexible tube taken along line A-A shown in FIG. 3;

FIG. 8 is a cross-sectional view of a flexible tube equipped with plurality of sensors;

FIG. 9 is a front view of the monitor shown in FIG. 2;

FIG. 10 is an enlarged view of a indicator shown in FIG. 9;

FIG. 11 is a front view of the monitor displaying a numeric value indicating an inserted length of the flexible tube;

FIG. 12 is a front view of the monitor displaying the insertion length indicator of the flexible tube overlaid with a pattern diagram of a human body;

FIG. 13 is a flowchart illustrating an inserted length displaying procedure when sensors are temperature sensors;

FIG. 14 is a flowchart illustrating an inserted length displaying procedure when the sensors are pressure sensors or vibration sensors;

FIG. 15 is a flowchart illustrating an inserted length displaying procedure in which the inserted length is detected in accordance with a difference between values measured by adjoining sensors;

FIG. 16 is a cross-sectional view of a flexible tube equipped with plurality of sensors;

FIG. 17 is an enlarged view of an indicator shown in FIG. 9;

FIG. 18 is a flowchart illustrating an inserted length displaying procedure when the sensors are temperature sensors;

FIG. 19 is a cross-sectional view of a flexible tube equipped with a plurality of types of sensors;

FIG. 20 is a flowchart illustrating an inserted length displaying procedure when temperature sensors, optical sensors, pressure and vibration sensors, wetness sensors, and humidity sensors are used; and

FIG. 21 is a diagrammatic view of a configuration of an fiber-optic endoscope according to an embodiment of the present invention.

### DETAILED DESCRIPTION OF THE EMBODIMENT

Referring to the accompanying drawings, endoscope systems according to embodiment and modifications of the present invention will be described in detail.

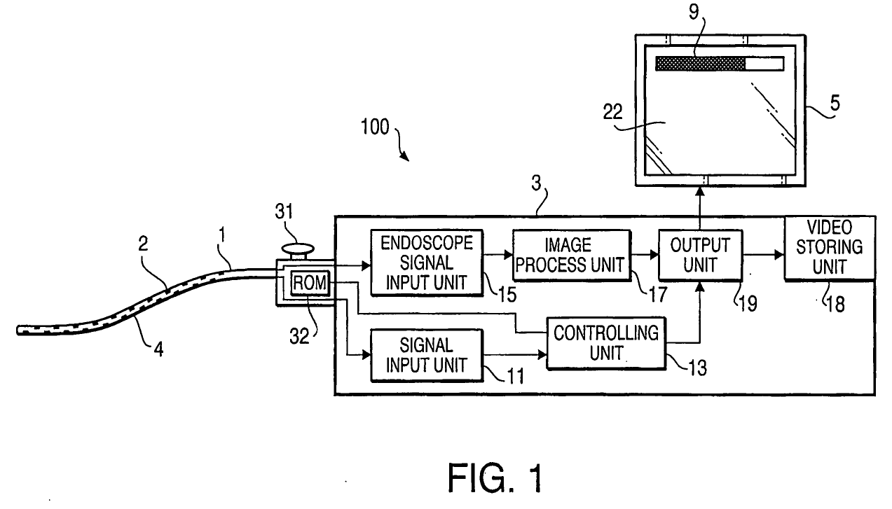

FIG. 1 is a diagrammatic view of a configuration of an endoscope system 100 according to an embodiment of the present invention. The endoscope system 100 is provided with an endoscope 1 for observing objective (affected) area of a patient, a processor 3 that processes various data, and a monitor 5 for displaying information such as images through the endoscope 1. The endoscope 1 is provided with a flexible tube 4 that is to be inserted into the body cavity of the patient and an operation unit 31 that is used for operating the endoscope 1. At the distal end of the endoscope 1, there is provided a distal section (not shown) wherein an image sensing unit (not shown) is installed to capture images inside the body of the patient. In addition, a plurality of sensors 2 for measuring environmental conditions surrounding the endoscope 1 are provide to the flexible tube 4. For these sensors 2, various types of sensors including temperature sensors, optical sensors, pressure sensors, vibration sensors, wetness sensors (i.e. electrodes), and humidity sensors may be used individually or in combination. The sensors 2 are arranged in the axial direction of the endoscope 1 to detect environmental conditions at respective sensors 2. Since the sensors 2 provided to the inserted portion of the endoscope 1 and those provided to the not-inserted portion of the endoscope 1 indicate different detection results, if the outputs of respective sensors 2 are identified, and the location of each sensor 2 (e.g., a length from the distal end of the endoscope 1 to each sensor 2) is known, the length of the inserted portion of the endoscope 1 can be obtained based on the outputs of the sensors 2.

The processor 3 is provided with a sensor signal input unit 11 to receive signals obtained from the sensors 2, a controlling unit 13 to process the signals obtained from the sensors 2, an endoscope signal input unit 15 to which image signals output by the image sensing unit is inputted, an image process unit 17 that converts the image signals to video signals, an output unit 19 to output the video signals to the monitor 5, and a video data storing unit 18 to store the output of the output unit 19 as video data The monitor 5 displays an image 22 based on the video signal output by the endoscope 1 and an insertion length indicator 9 to indicate the inserted length of the endoscope 1 (i.e. the flexible tube 4, more specifically).

The process implemented by the endoscope system 100 will be described below. An optical image of the object inside a human body is formed on an image receiving area of the image sensing unit. The image signals are then inputted to the endoscope signal input unit 15, successively transmitted to the image process unit 17 to be converted as video signals. Then the video signals are transmitted via the output unit 19 to the monitor 5 to be displayed as the images 22 from the endoscope 1. In addition, the video signals are sent to the video storing unit 18 and stored, so as to be reviewed afterward. The operation unit 31 is configured such that an observing areas can be adjusted by rotating a rotator that modifies flexion of the endoscope 1. The operation unit 31 is provided with buttons that enable various operations including switching ON/OFF of indication of the insertion length indicator 9 and inputting of various data manually.

Signals obtained by the sensors 2 are inputted to the sensor signal input unit 11, then sent to the controlling unit 13. In the controlling unit 13, the signals are processed to determine the inserted length of the endoscope 1. Then, based on the inserted length thus determined, information to be displayed on the monitor 5 is generated. The information to be displayed as well as the inserted length thus obtained are transmitted via the output unit 19 to the monitor 5.

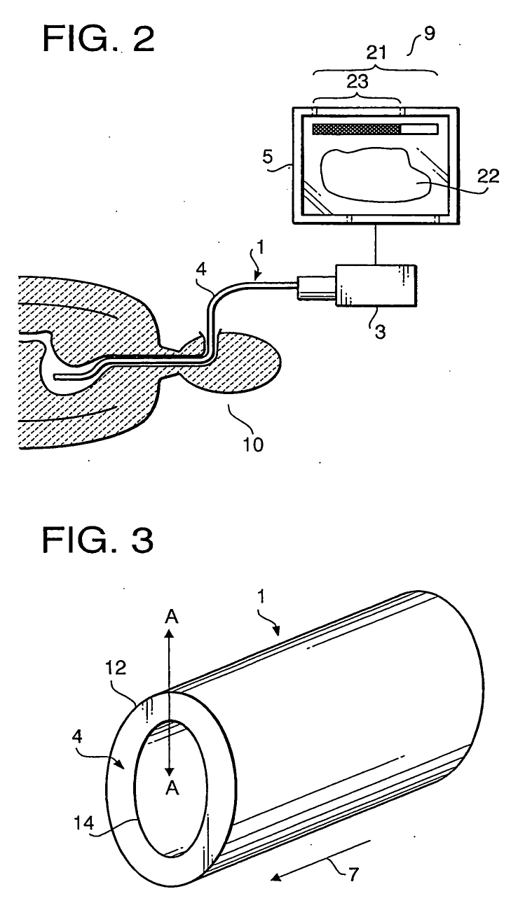

FIG. 2 schematically shows a configuration of the endoscope system 100. As shown in FIG. 2, the flexible tube 4 of the endoscope 1 is inserted in a human body 10. The insertion length indicator 9 displayed on the monitor 5 is a so-called thermometer type indication, which includes an endoscope full-length indicator bar 21 and an insertion length indicator bar 23 that indicates the length of the flexible tube 4 inserted into the body. Specifically, the endoscope full-length indicator bar 21 is a white bar representing the full length of the flexible tube 4, and the insertion length indicator bar 23 is a black bar overlapping the full-length indicator bar 21. As will be described later, when the inserted length of the flexible tube 4 obtained based on the signals from the sensors 2, the insertion length indicator bar 23 having a length corresponding to the inserted length is overlaid on the endoscope full-length indicator 21 as the blackened bar on the monitor 5.

FIG. 3 is a perspective view of an portion of the flexible tube 4 of the endoscope 1. An arrow 7 indicates a direction along which the flexible tube 4 (endoscope 1) is inserted in the human cavity. The flexible tube 4 has an outer surface 12 exposed to outside (an environment surrounding the flexible tube 4) and an inner surface 14.

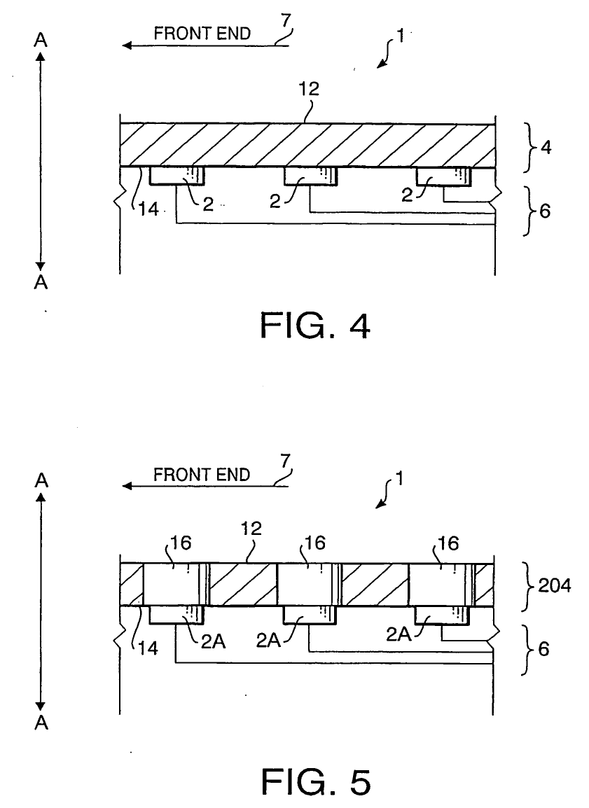

FIG. 4 is a cross-sectional side view of the portion of the flexible tube taken along line A-A in FIG. 3. Corresponding to FIG. 3, the arrow 7 indicates a direction along which the flexible tube 4 is inserted in the human cavity. The endoscope 1 is provided with the sensors 2, the flexible tube 4 and sensor cables 6. The sensors 2 are arranged along a longitudinal axis of the endoscope 1 (the flexible tube 4), with predetermined interspaces therebetween. Each sensor 2 is connected to a sensor cable 6, which is connected to the sensor signal input unit 11 of the processor 3.

The process implemented by the endoscope system when the sensors 2 are temperature sensors will be described below. Generally, temperature in an operation room for endoscopic observation is maintained at an equivalent level to a normal room temperature. When the endoscope 1 is inserted into the human body, the values obtained from the temperature sensors 2, which are arranged on the inserted portion of the flexible tube 4 increase according to the body temperature, which is generally higher than the room temperature. On the other hand, other temperature sensors 2 arranged on the other portion of the flexible tube 4 which is not inserted into the body, are not affected by the body temperature, and are maintained at the room temperature level. As described below, the sensors 2 are sequentially numbered from the distal end toward the proximal end of the flexible tube 4 along the longitudinal axis, so that the location of a sensor 2 can be specified by the number thereof. By referring to the thus assigned sensor numbers, an amount of the insertion portion with respect to the entire length of the flexible tube 4 can be obtained easily as described later. Further, the actual length of the inserted portion can also be obtained easily. That is, the length of the inserted portion of the flexible tube 4 can be determined by identifying the temperature sensor located is at a “boundary” between the sensors that are affected by the body temperature and the other sensors that are maintained at the room temperature.

First Modification

The process implemented by the endoscope system when the sensors 2 are optical sensors 2 will be described below. Generally, quantity of light in an operation room for endoscopic observation is maintained at an equivalent level to a normal room lightness. Inside a body, on the other hand, is dark with no lighting source. When the flexible tube 4 is inserted into a body, the optical sensors 2, even arranged inside the flexible tube 4, are capable of sensing light, therefore it is possible to determine that the portion with optical sensors 2 sensing more light is outside the body, while the portion with optical sensors 2 sensing less light is inside. Thus, the length of the inserted portion of the flexible tube 4 can be determined by specifying which optical sensor 2 is at a boundary between the sensors 2 that are sensing more light and the sensors that are sensing less light. Generally, an endoscope with optical sensors are more responsive than an endoscope with temperature sensors 2. Therefore, the endoscope provided with the optical sensors 2 is particularly useful when the flexible tube 4 is moved relatively quickly.

Although the flexible tube 4 is capable of transmitting sufficient light for determining the inserted length of the flexible tube 4, it is possible to configure the flexible tube 4 to increase the quantity of light to be sensed by the optical sensors 2.

FIG. 5 shows a cross-sectional view of the flexible tube 4 of the endoscope 1 taken along line A-A of FIG. 3. Sensors 2A are optical sensors, while a flexible tube 204 is provided with optically transmittable flexible members 16. The other configurations are similar to those shown in FIG. 4, description thereof will be omitted for brevity. The sensors 2A are arranged on the inner surface 14 of the flexible tube 4, in contact with the flexible members 16, respectively. Alternatively, the entire flexible tube 4 may be configured with optically transmittable material.

Second Modification

The process implemented by the endoscope system when the sensors 2 are pressure sensors or vibration sensors will be described below (see FIG. 4). In this configuration, the length of the flexible tube 4 inserted in a body is obtained by detecting vital activities such as heartbeat or respiration. The environmental changes in vibration or pressure caused by heartbeat or respiration are more significant inside the body than outside. It is known that the frequency of heartbeat is in or around a range from 60 to 100 times per minute. Therefore, based on whether the frequencies are higher than predetermined levels, for example from 1 (i.e. 60 times divided by 60 seconds) to 1.67 (i.e. 100 times divided by 60 seconds), it is possible to determine whether a portion of the flexible tube 4 is inside or outside the body.

Waveforms of heartbeats consist of various waves, such as P wave, QRS wave, T wave, and U wave. Among these waves, for example, QRS wave is easier to be sampled than other waves, as a set of QRS wave generally forms higher and more distinct peaks than others. Therefore, in this embodiment, a frequency indicating the peak values is obtained from sampled QRS wave. Also, a set of QRS wave takes a range over the time axis, generally from 0.06 second to 0.10 second. For obtaining a frequency indicating peak values, an interval to be sampled must be short enough, that is less than the range (i.e. from 0.06 to 0.10). Thus, in this embodiment, an interval to be sampled is set to 0.03 second, which is a half of 0.06 second.

For calculating the inserted length of the flexible tube 4, sampled data is analyzed by Fast Fourier Transform (hereinafter referred as FFT). For FFT, 2ⁿ pieces of data must be sampled (“n” is an arbitrary natural number). The spectrum resolution resulted from FFT increases as the value of n increases. However, when n is increased, data to be sampled also increment, resulting to take longer time to obtain the frequency indicating the peak values. Considering these limitations, the number of data sampled in one measurement is set to 64 (i.e. 2⁶) in the present embodiment. With this number, the spectrum resolution can be maintained relatively high, while the measurement is performed in a short period (i.e. 0.03*64=1.92 seconds). Based on the measured result, the controlling unit 13 determines that the sensors with the frequency between 1 Hz and 1.67 Hz are inside the body. Also, the sensors with the frequency outside the range between 1 Hz and 1.67 Hz are determined to be outside the body. With this determination, the sensor at the boundary is specified, and the inserted length of the flexible tube 4 is calculated.

Third Modification

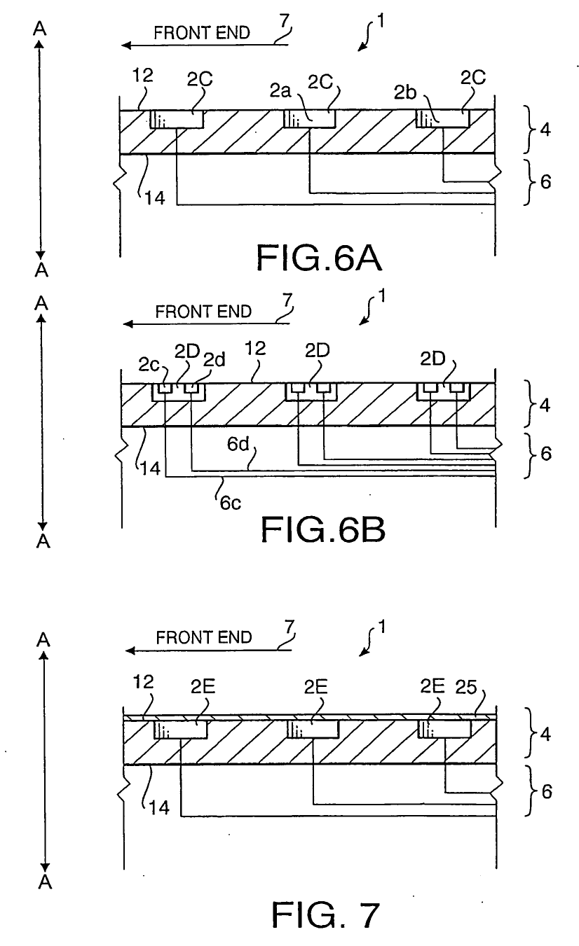

The process implemented by the endoscope system when the sensors 2 are wetness sensors 2C will be described below. FIG. 6A is a cross-sectional view of a portion of the flexible tube 4, taken along line A-A of FIG. 3, when wetness sensors 2C are employed. The wetness sensors 2C are arranged to be exposed to the outside environment surrounding the flexible tube 4. One sensor 2C includes an electrode 2a and another sensor 2C includes electrode 2b, which are alternately arranged in the axial direction of the flexible tube 4. Each of the electrodes 2a and 2b are connected to the sensor signal input unit 11 through the cables 6, respectively. Two adjoining sensors 2C detect the wet condition as the electrodes 2a and 2b are electrically connected by wet substance such as mucous membrane or the like covering the electrodes 2a and 2b. For the other configurations, explanation will be omitted, as they are similar to the configurations drawn in FIG. 4.

When the endoscope 1 is inserted into a body, the wetness sensors 2C become wet by mucous membrane, digestive fluid in digestive canals or the like, and the electrode 2a and electrode 2b are electrically connected. Since the wetness sensors 2C outside the human body will not be wet, the location of the sensors 2C can be determined depending on whether the electrodes 2a and 2b are electrically connected.

Fourth Modification

FIG. 6B shows another example of wetness sensors 2D. Each wetness sensor 2D includes at least two electrodes 2c and 2d arranged in the axial direction of the flexible tube 4. In addition, the electrode 2c and the electrode 2d are connected to a sensor cable 6c and a sensor cable 6d, respectively. Similar to the third modification, the electrically connected electrodes 2c and 2d on the proximal end side are the electrodes 2c and 2d represent the proximal side end position inside the human body 10 of the flexible tube 4. Since the other configurations are similar to those of the third modification, a further description will be omitted. It should be noted that, although in FIG. 6B the electrode 2c and the electrode 2d are arranged in the axial direction of the flexible tube 4, these electrodes may be arranged in the circumferential direction in another embodiment.

Fifth Modification

The process implemented by the endoscope system when the sensors are humidity sensors 2E will be described below. FIG. 7 is a cross-sectional view of a portion of the flexible tube 4 taken along line A-A of FIG. 3. A layer 25 is a water-repellent layer which allows water moisture, but prevents normal water from passing therethrough.

Generally, relative humidity in an operation room for endoscopic observation is normal room humidity, which is within a range from 30% to 80%, while the humidity inside a human body is close to the saturated water vapor pressure (i.e., approximately 100%). Therefore, based on whether the measured humidity is approximately 100% or not, it is determined whether the each sensor 2E is inside or outside of the body. Since the humidity sensors 2E must contact the gas inside the digestive canals, the sensors 2E are exposed to the environment outside the flexible tube. However, the humidity cannot be measured when the surface of the sensors 2E are wet. Accordingly, as shown in FIG. 7, the surface of the humidity sensors 2E covered with the water-repellent layer 25.

In order to identify the sensors 2 inside/outside the human cavity, the sensors 2 are numbered. Hereinafter, a numbering method to number the sensors 2 will be described.

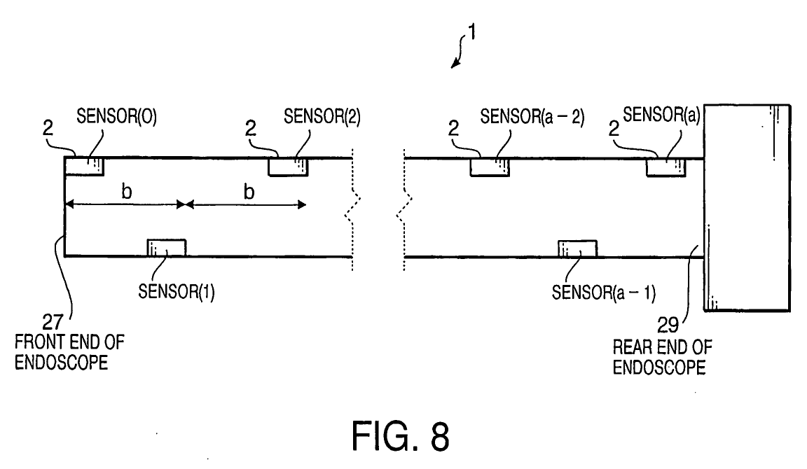

FIG. 8 is a cross-sectional view of an endoscope 1, equipped with plurality of sensors 2. In FIG. 8, the flexible tube 4 is schematically depicted for explanation purpose. The front end (distal end) 27 of the endoscope 1 is on the left-hand end of the longitudinal axis of the endoscope 1, and the rear end (proximal end) 29 is on the right-hand end of the longitudinal axis. The sensors 2 are arranged sequentially from the distal end 27 toward the proximal end 29, with a predetermined interspaces between each other. The sensors 2 are arranged to be substantially equally spaced in the present embodiment and modifications, however, the interspaces may be adjusted respectively according to conditions.

As shown in FIG. 8, the sensor 2 that is closest to the distal end 27 is numbered as the sensor (0), and the successive sensors 2 are accordingly numbered sequentially up to the last sensor (a), which is closest to the proximal end 29. In addition, the sensor (0) is provided at the distal end 27. The sensor (1) is arranged in a position where the proximal end of the sensor (I) corresponds to the end of a length b. The succeeding sensors 2 are arranged in positions where the rear end of the sensors 2 correspond to the end of lengths b from the preceding sensors 2. These conditions defined above are to be considered only for the convenience of explanation, and the scope of the present invention is not restrictive to only these conditions.

In FIG. 8, the sensors 2 are arranged alternately. That is, two successive sensors 2 are arranged at opposite positions in the circumferential direction, and spaced in the axial (i.e., longitudinal) direction. By this arrangement, the sensors 2 can be closely arranged with less interspaces between each other, therefore accuracy to measure the inserted length is improved. It should be noted that the arrangement of the sensor 2 need not be limited to the configuration shown in FIG. 8, which only shows an exemplary arrangement, and the sensors 2 may be arranged in a linear alignment, a spiral alignment, or combination thereof.

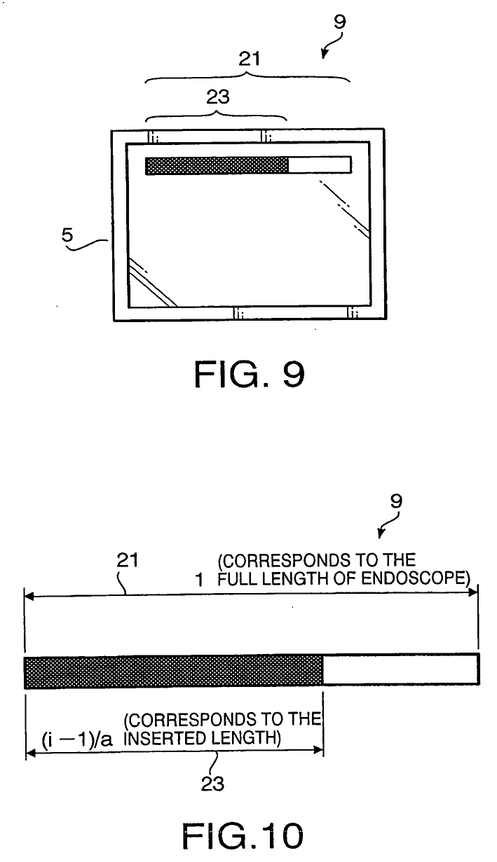

A configuration of images indicating the length inserted to the endoscope 1, which is displayed on the monitor 5 will be described below. FIG. 9 is an enlarged view of the monitor 5 shown in FIG. 2. The insertion length indicator 9 displayed on the monitor 5 is configured with the endoscope full-length indicator bar 21 and the insertion length indicator bar 23. The endoscope full-length indicator bar 21 indicates the full length of the endoscope, which is the length from the distal end 27 to the sensor (a) (see FIG. 8). The insertion length indicator bar 23 indicates the length from the distal end 27 to the sensor (i−1) that is at the boundary between the inside and the outside of a body. In this situation, the sensor (i) is at the closest position to the distal end 27 among all other sensors that are considered to be outside the body. In this embodiment, the endoscope 1 comprises a ROM 32, wherein, for example, information on the sensors is stored. The information on the sensors includes, for example, type of the sensors, number of the sensors, and interspaces between the sensors. When the controlling unit 13 of the processor 3 is connected to the endoscope 1, the controlling unit 13 obtains the information from the ROM 32 to calculate the full length of the endoscope 1 (more specifically, the portion of the endoscope 1 inserted into the body, which is the full length of the flexible tube 4).

A method of displaying the insertion length indicator 9 will be described. FIG. 10 is an enlarged view of the insertion length indicator 9 shown in FIG. 9. Throughout the endoscope 1, there are provided “a” pieces of interspaces between the sensors 2 (“a” is the number of the sensor positioned at the closest to the proximal end 29). In addition, from the distal end 27 to the sensor (i−1), which is closest to the proximal end 29 among the other sensors that are inserted into a body, there are provided “i−1” pieces of interspaces between the sensors 2 that are inserted into a body. Thus, when the length of the endoscope full-length indicator bar 21 is presented as 1, the length of the insertion length indicator bar 23 is indicated as (i−1)/a. Then, with the actual full length of the endoscope 1, which is generally known, the numeric value to be indicated as the insertion length is calculated easily as follows: The actual full length of the endoscope 1 is multiplied by (i−1)/a to be the numeric value that is to be indicated as the inserted length of the endoscope 1.

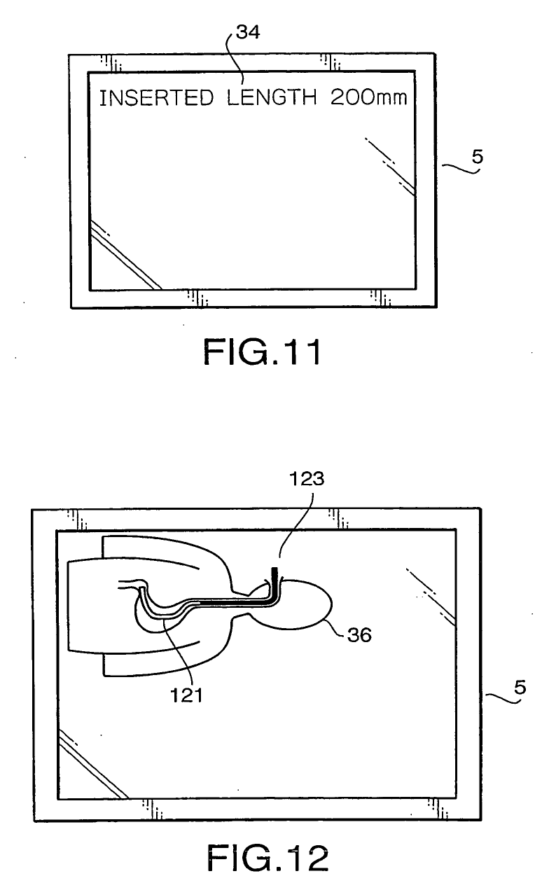

The process to display the value indicating the inserted length of the endoscope 1 will be described. FIG. 11 is a diagrammatic view of the monitor 5 displaying a numeric value indicating the inserted length of the flexible tube 4. As described above, the actual value to be indicated by the insertion length indicator bar 23 is easily calculated, and displayed on a numeric display 34 of the monitor 5. An operator can switch the images to be displayed on the monitor 5 from the insertion length indicator 9 to the numeric display 34, and vice versa. Thus, the actual numeric value of the insertion length can be confirmed easily on the monitor 5. Alternatively, the numeric display 34 may be displayed along with the insertion length indicator 9 on the monitor 5.

The process to display a pattern diagram of a human body overlaid with an insertion length indicator will be described. FIG. 12 is a diagrammatic view of an insertion length indicator bar 123 of the flexible tube 4 overlaid with a pattern diagram of a human body 36. As shown in FIG. 12, an endoscope full-length indicator bar 121 is embedded in the pattern diagram of a human body 36. Both the pattern diagram of a human body 36 and the endoscope full-length indicator bar 121 are stored in the processor 3. When the flexible tube 4 is inserted into a body, the insertion length indicator bar 123 corresponding to the actual inserted length is overlaid on the endoscope full-length indicator bar 121 as a blackened portion. With this configuration, the insertion state of the endoscope 1 can be recognized easily. Optionally, a body height of a patient may be inputted, in order to adjust the relationship between the pattern diagram of a human body 36 and the full length of the flexible tube 4 on the monitor 5.

A control flow to measure the actual length of the inserted portion of the flexible tube 4 will be described below.

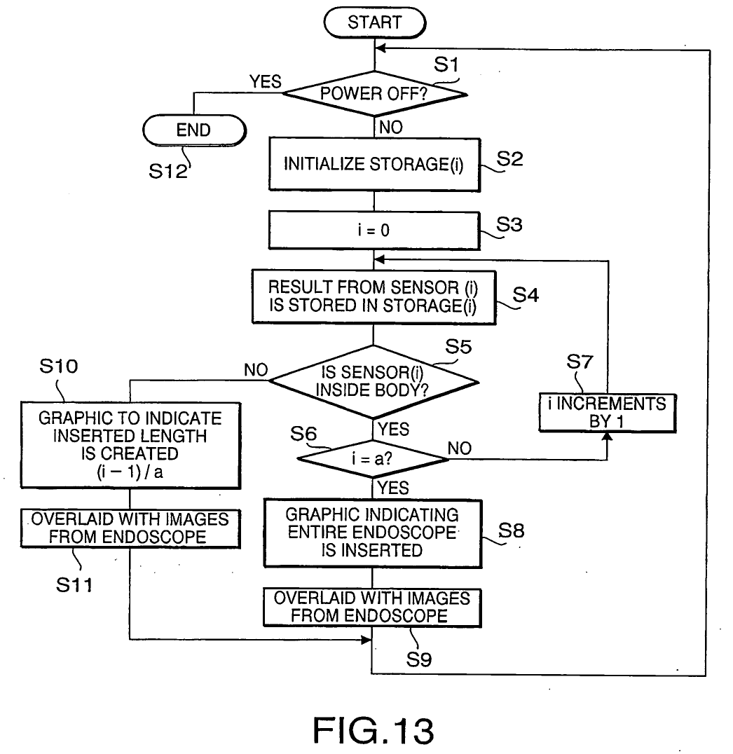

As an example, a control flow, when the sensors 2 are temperature sensors, will be described. FIG. 13 is a flowchart showing a control flow of displaying a measured inserted length of the flexible tube 4 on the monitor 5, when the sensors 2 are temperature sensors.

First, process checks whether the power supply is turned off (S1), and when it is not turned off (S1: NO), process proceed to S2. In this procedure, a value of the sensor(i) is obtained and stored in a storage(i) of a memory. In S2, all the storages, i.e., storage(i) for i=0 to i=a, are initialized. That is, all the storages(i) are set to zero. Hereinafter, the value stored in the storage(i) is referred as {storage(i)}. In S3, a variable i is set to zero. Then, starting from the sensor(i) (i.e., sensor(0)), which is at the distal end of the flexible tube 4, the measured values is obtained and stored in storage(i). Hereinafter i is referred to as a number that is greater than 0, on the assumption that the steps from S5 to S7 (described later) have been already performed several times. In S4, after the measured result is obtained, the measured value of the sensor(i) is stored in the storage(i). Then, in S5, it is determined whether the portion at which the sensor(i) is located is inside the human body. Specifically, when the sensors 2 are the temperature sensors, {storage(i)} is compared with a predetermined value, in S5. The predetermined value is assumed to be approximately equivalent to a normal body temperature, for example 37 degrees C. If the measured temperature value stored in the storage(i) is greater than the predetermined value (S5: YES), it is considered that the portion of the flexible tube 4 at which the sensor(i) is located is inside the body. Therefore, process proceeds to S6. In S6, process judges whether the sensor(i) is the sensor(a), which is arranged closest to the proximal end. That is, process judges whether temperature values of all the sensors have been read and stored in the storage(i). If the sensor(i) is not the sensor(a) (S6: NO), process proceeds to S7, and i is incremented by 1. Then, process proceeds to S4.

In S5, if control determines that {storage(i)} is equal to or smaller than the predetermined value, process proceeds to S10. It should be noted that, if the temperature value stored in the storage(i) is not greater than the predetermined value, it is considered that the portion of the flexible tube 4 at which the sensor(i) is located is outside the body. Accordingly, when process proceeds from S5 to S10 when {storage(i)} is checked, it is known that the values stored in storage(0)−storage(i−1) are greater than the predetermined threshold value, while {storage(i)} is not greater than the predetermined value. Therefore, the sensor(i−1) is the sensor located at the proximal side end among the sensors inside the body cavity, while the sensor(i) is the sensor located at the distal end side among the sensors outside the human body. Accordingly, in S10, by calculating (i−1)/a, the inserted amount is obtained, and a graphic image (the insertion length indicator 9) to indicate the inserted length is generated. In S11, the created image of the insertion length indicator 9 is overlaid with an observation image 22 captured by the endoscope 1, on the monitor 5. Then the process returns to S1 to repeat the above-described steps to refresh the image of the insertion length indicator 9. Further, the image of the insertion length indicator 9 created in S 11 continues being overlaid until the next image is created.

In S6, process judges whether the sensor(i) is the sensor(a), that is, whether all the sensors have been checked. If the sensor(i) is the sensor(a) (S6. YES), an image of the insertion length indicator 9 in which the insertion length indicator bar 23 overlaps the full-length indicator bar 21 is generated (S8). In S9, the created image is overlaid with the image 22 captured by the endoscope 1 on the monitor 5. Then process returns to S1 to repeat the steps described above. The created image continues being overlaid until the next image is created. If process receives a command to turn off the power supply, the measurement is terminated (S12). It should be noted that although the results from each sensor are obtained and judged sequentially in the present embodiment, the results from all the sensors may be obtained collectively and judged in another embodiment.

If the optical sensors are used as the sensors 2, in S5 of the flowchart shown in FIG. 13, process judges whether the output of the sensor(i) is smaller than a predetermined value. It should be noted that, a sensor outside the body cavity receives more light than a sensor inside the body cavity. Thus, by comparing the output value of the sensor(i) with the predetermined value, whether the sensor(i) is outside/inside the body cavity is known. In S5 of FIG. 13, when the sensors 2 are optical sensors, control proceeds from S5 to S6 if the output value of the sensor(i) (i.e., {storage(i)}) is smaller than a predetermined value, while if the {storage(i)} is not smaller than the predetermined value, control proceeds from S5 to S10.

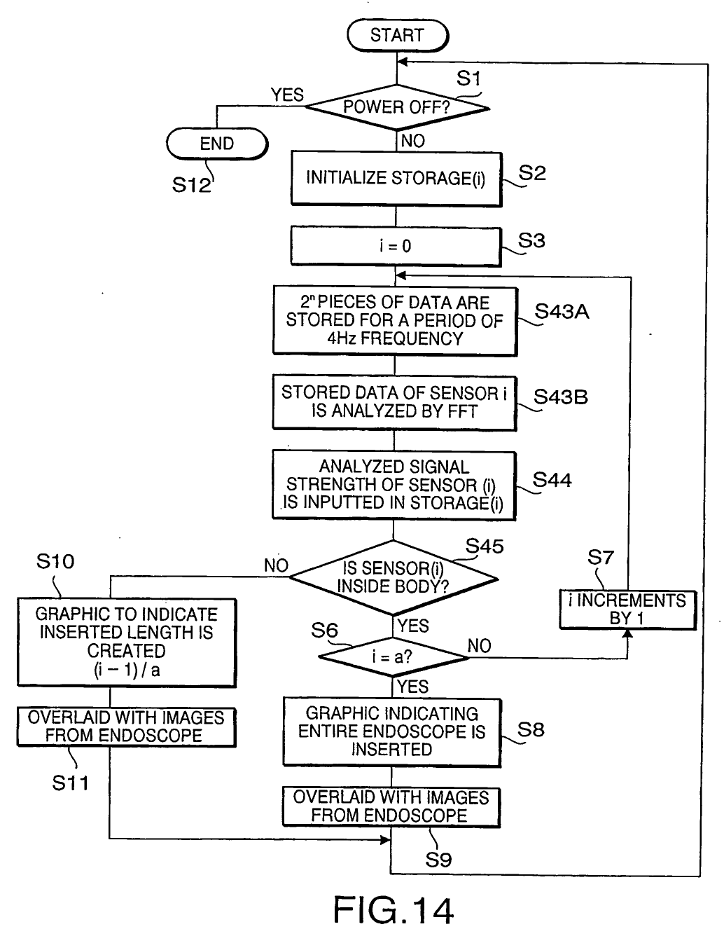

FIG. 14 is a flowchart showing a control flow of displaying a measured inserted length of the flexible tube 4 on the monitor 5, when sensors 2 are pressure sensors or vibration sensors. FIG. 14 is similar to FIG. 13 except that steps S4 and S5 have been changed to steps S43A-S44 and S45. In S43A, 2ⁿ pieces of data of the sensor(i) are sampled for a predetermined period of time. In S43B, the sampled data is analyzed by FFT to detect a peak value of the frequency. In S44, the obtained peak value is stored in storage(i). In S45, the value stored in storage(i) (i.e. {storage(i)}) is judged whether it is in a range between 1 Hz and 1.67 Hz. If {storage(i)} is not in this range (S45: NO), process proceeds to S10. If {storage(i)} is in the range (S45: YES), process proceeds to S6.

Next, a control flow implemented by the endoscope system when the sensors 2 are wetness sensors shown in FIG. 6B will be described with reference to FIG. 13.

In this case, the sensors 2D outputs one (conducted: wet) or zero (non-conducted: not wet). When the sensor(i) is inside a body, the electrode 2c and the electrode 2d provided to the sensors 2 are conducted, or electrically connected (see FIG. 6B). In STEP 6, if the sensor(i) is conducted, a value 1 is stored in storage(i), while if the sensor(i) is not conducted, a value 0 is stored in storage(i). In S5, process judges whether {storage(i)}is 1 or 0. If {storage(i)} is equal to 1 (S5: YES), the portion of the flexible tube 4 at which the sensor(i) is located is considered to be inside the human body, and process proceeds to S6. If {storage(i)} is not equal to 1 (S5: NO), the sensor 2D is outside the human body, and process proceeds to S10.

The control flow implemented by the endoscope system when the sensors 2 are wetness sensors, which are the wetness sensor 2a and the wetness sensor 2b shown in FIG. 6A, will be described below.

When sensor(i) is inside a body, the inserted sensor(i−1) and the sensor (i) become wet, and the electrode 2a and the electrode 2b which are provided as different sensors 2 are conducted (refer to FIG. 6A). If sensor(i−1) and sensor(i) are conducted, a value 1 is stored in storage(i), and if sensor(i−1) and sensor (i) are not conducted, a value 0 is stored in storage(i).

The control flow implemented by the endoscope system when the sensors 2 are humidity sensors will be described with reference to FIG. 13.

Generally, relative humidity in an operation room for endoscopic observation is normal room humidity, which is from 30 to 80%, while the humidity in a human body is close to 100%. Therefore, the boundary is judged by determining whether the relative humidity is close to 100% or not. In S5, if {storage(i)} is equal to or smaller than a predetermined value (S5: YES), it is judged that sensor(i) is outside the human body, and process proceeds to S10. If {storage(i)} is greater than the predetermined value (S5: NO), it is judged that sensor(i) is inside the human body, and process proceeds to S6. For the predetermined value, any value close to the relative humidity of nearly 100% is selected.

Sixth Modification

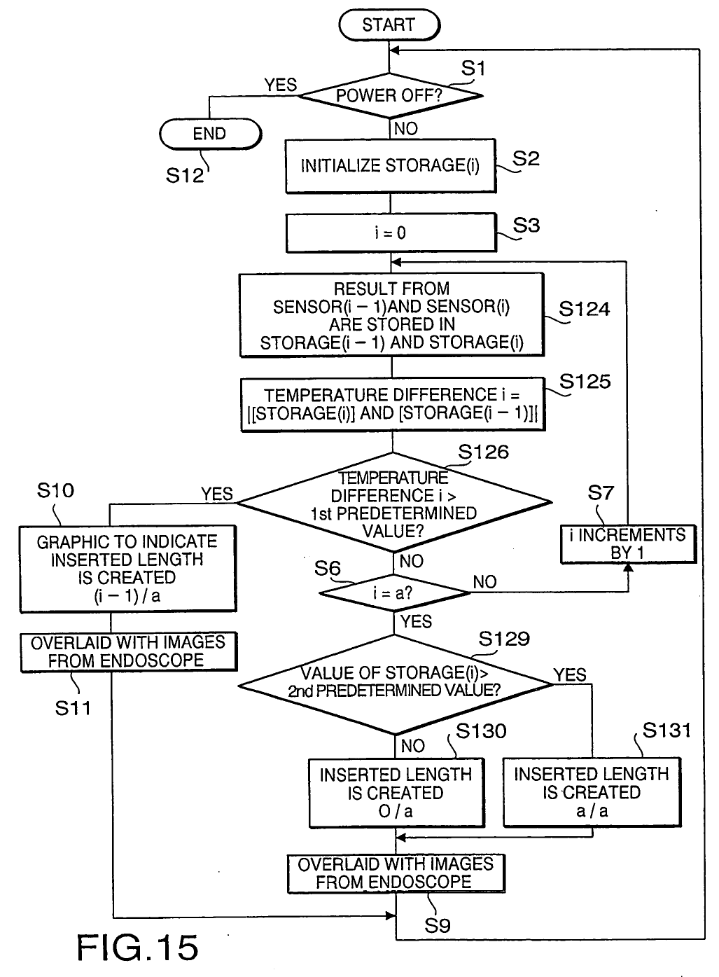

Referring to FIG. 15, a procedure for detecting and displaying an inserted length of the flexible tube 4 according to a sixth modification will be described. In this modification, a boundary between the inserted portion and outside portion of the flexible tube 4 is identified by comparing predetermined value with a difference between measured values of sensor (i−1) and adjoining sensor (i). Hereinafter, description will be made on assumption that the sensors 2 are temperature sensors, although the following description is applicable to optical sensors, pressure sensors, vibration sensors, and humidity sensors.

FIG. 15 is a flowchart showing a procedure of displaying the insertion length indicator 9 based on a difference between values measured by sensor (i) and adjoining sensor (i−1) and the predetermined value.

In S124, a measured result of the sensor (i−1) and another measured result of the sensor (i) are stored in storage(i−1) and storage(i), respectively. In S125, a difference between the values stored in storage(i−1) and {storage(i)} is calculated, and the absolute value thereof is derived, which is referred to as a Temperature Difference(i). In S126, process judges whether the Temperature Difference(i) is greater than a first predetermined value. If the Temperature Difference(i) is greater than the first predetermined value (S126: YES), process proceeds to S10. If the Temperature Difference(i) is not greater than the first predetermined value, process proceeds to S6. In S6, if i is not equal to “a” (S6: NO), the process proceeds to S7. If the variable i is equal to “a” (S6: YES), the process proceeds to S129.

In S129, {storage(i)} is compared with a second predetermined value. In this modification, as the second predetermined value, a temperature approximately equivalent to a normal body temperature, for example 37 degrees C., is used. If {storage(i)} is greater than the second predetermined value (S129: YES), the full length of the flexible tube 4 is considered to be inserted into the body, and process proceeds to S131. In S131, the insertion length indicator 9 indicating that the entire flexible tube 4 is inserted is created. If {storage(i)} is not greater than (i.e., equal to or smaller than) the second predetermined value (S129: NO), no portion of the flexible tube 4 is considered to be inserted. In this case, process proceeds to S130, where the insertion length indicator 9 indicating that no portion of the flexible tube 4 is inserted is created.

Seventh Modification

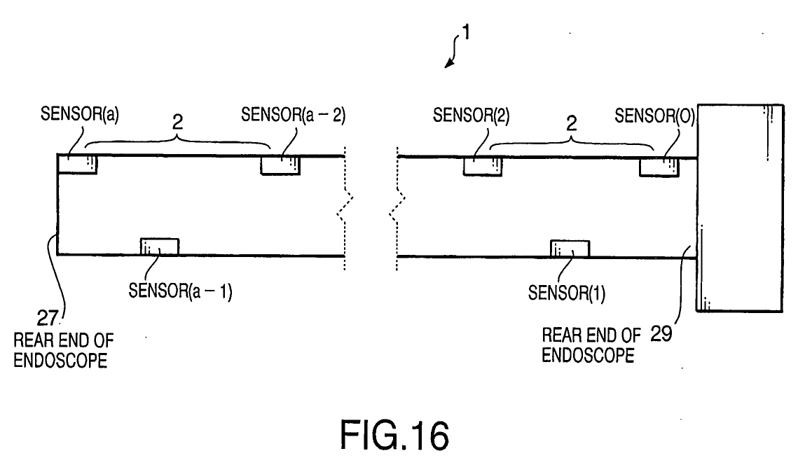

In the embodiment and modifications described above, the sensors 2 are numbered such that one closest to the distal end 27 is numbered as the sensor(0), and the other sensors 2 are numbered sequentially up to the last sensor(a), which is closest to the proximal end 29. The invention need not be limited in such configurations, and the sensors 2 may be numbered in an opposite manner. That is, as shown in FIG. 16, the sensor 2 closest to the proximal end 29 may be numbered as sensor(0), and the other sensors 2 are numbered sequentially up to the last sensor(a), which is closest to the distal end 27.

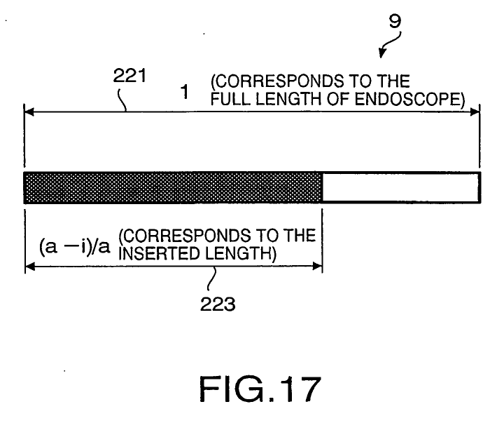

A method of displaying the insertion length indicator 9 when the sensors 2 are numbered as above will be described. FIG. 17 illustrates an enlarged view of the insertion length indicator 9 according to the seventh modification. The insertion length indicator 9 displayed on the monitor 5 is configured with the endoscope full-length indicator bar 221 and the insertion length indicator bar 223. The endoscope full-length indicator bar 221 indicates the full length of the endoscope 1, from the distal end 27 to the sensor(a) (see FIG. 16). The insertion length indicator bar 223 indicates the length from the distal end 27 to sensor(a−i) that is the proximal end side one among the sensors 2 inside the human body. In this situation, sensor(i) is the distal end side one among the sensors outside the body.

Throughout the flexible tube 4, there are provided “a” pieces of interspaces between the sensors 2 (“a” is the sensor number of the distal end side sensor). Throughout the inserted portion of the flexible tube 4, there are provided “a−i” pieces of interspaces between the sensors 2. Thus, when the length of the endoscope full-length indicator bar 221 is presented as 1, the length of the insertion length indicator bar 223 is indicated as (a−i)/a. Since the actual full length of the flexible tube 4 is known, the numeric value of the insertion length can be calculated easily by multiplying the full length of the flexible tube 4 by (a−i)/a

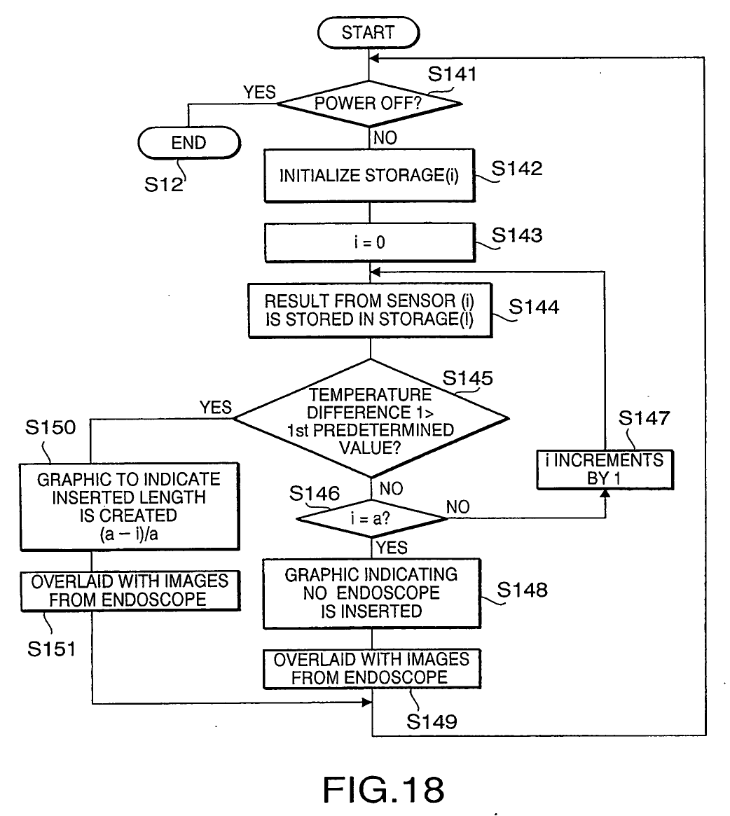

FIG. 18 is a flowchart illustrating a procedure of displaying an inserted length of the flexible tube 4 on the monitor 5 according to the seventh embodiment, when the sensors 2 are temperature sensors. The procedure is similar to that shown in FIG. 13 except that steps 5, 8 and 10 have been replaced with steps 145, 148 and 150, respectively.

In S145, {storage(i)} is compared with a predetermined value. If {storage(i)} is not greater than the predetermined value (S145: NO), process proceeds to S146.

If {storage(i)} is greater than the predetermined value (S145: YES), process proceeds to S150. It should be noted that the sensor (i) is determined to be the closest sensor to the distal end 27 of the endoscope 1 among the other sensors 2 that are arranged on the portion that is not inserted in the body. Accordingly, the sensor 2 that is closest to the proximal end 29 of the flexible tube 4 among the other sensors 2 arranged on the inserted portion is the sensor (a−i). Thus, by calculating (a−i)/a, the inserted length can be obtained, and the image of the insertion length indicator bar 223 can be created.

If {storage(i)} is not greater than the predetermined value (S145: NO), the insertion length indicator 9 indicating that no portion of the flexible tube 4 is inserted is created (S148).

In each of the embodiment and modifications described above, only one type of the temperature sensors, optical sensors, pressure sensors, vibration sensors, wetness sensors, and humidity sensors is employed. The invention need not be limited to such a configuration, and more than one types of sensors may be employed in the same endoscope 1. In the following description, a method of displaying inserted lengths using two or more types of sensors will be described as an eight modification.

Eighth Modification

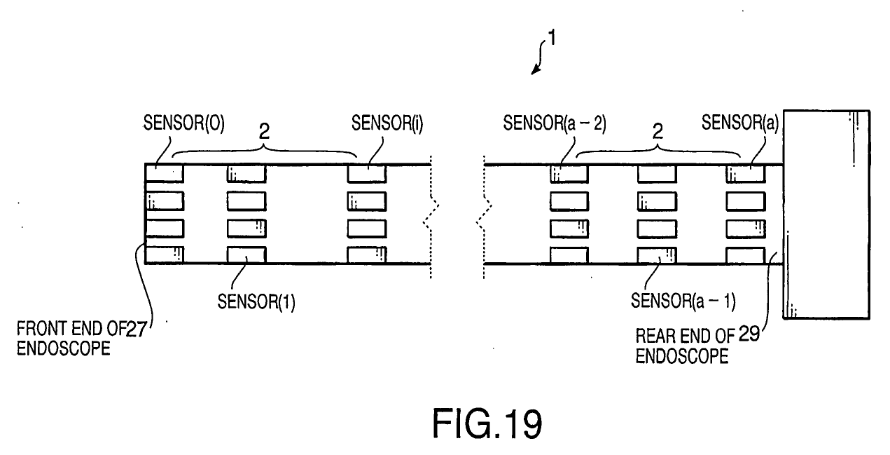

FIG. 19 is a cross-sectional view of an endoscope 1 according to an eighth modification, which is equipped with plurality of types of the sensors 2. The sensor groups 2G are numbered sequentially from the distal end 27 of the endoscope 1 according to a method shown in FIG. 8. Each of the sensor groups (i) includes a plurality of sensors 2, which are arranged in a circumferential direction, while the sensor groups 2G are arranged in the axial direction at predetermined interspaces therebetween. Each of the sensor groups 2G includes temperature sensor, optical sensor, pressure and vibration sensor, wetness sensor, and humidity sensor, which are either exposed to the outside environment surrounding the flexible tube 4 or embedded inside the surface of the flexible tube 4.

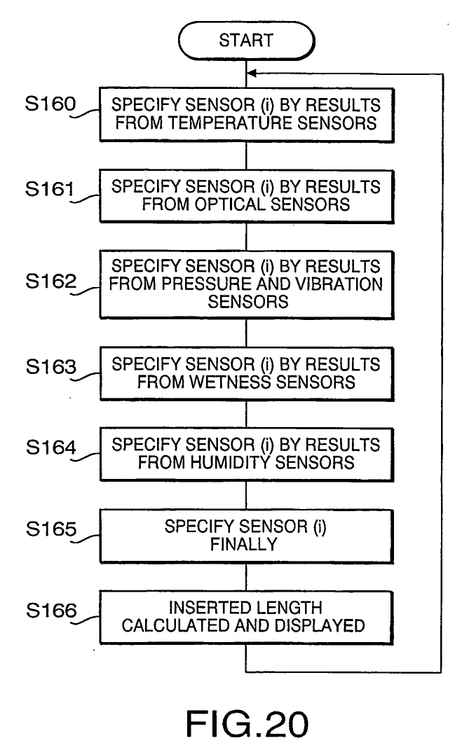

FIG. 20 is a flowchart showing a procedure for displaying the insertion length when the temperature sensors, optical sensors, pressure and vibration sensors, wetness sensors, and humidity sensors are used. In S160, among the temperature sensors, a sensor (i) at the closest position to the distal end 27 of the flexible tube 4 among ones considered to be outside the body, is identified. Next, among the optical sensors, a sensor (i) at the closest position to the distal end 27 of the endoscope 1 among ones considered to be outside the body, is identified in S161. In S162, among the pressure and vibration sensors, the sensor (i) at the closest position to the distal end 27 of the flexible tube 4 among ones considered to be outside the body, is identified. In S163, among the wetness sensors, a sensor (i) at the closest position to the distal end 27 of the flexible tube 4 among ones considered to be outside the body, is identified. In S164, among the humidity sensors, a sensor (i) at the closest position to the distal end 27 of the flexible tube 4 among ones considered to be outside the body, is identified. In should be noted that the method of identifying the sensor (i) outside the body is similar to the method aforementioned. In FIG. 20, the steps S160 to S164 are executed sequentially. This invention need not be limited to such a configuration, and more than one of the steps or all the steps may be executed parallelly.

In S165, the results obtained from each type of the sensors 2 (S160-S164) are analyzed. In this step, the sensor (i) at the closest position to the distal end 27 among all the other sensors that are outside the body is finally identified in accordance with a predetermined method, e.g., by comparing all the results aggregated from all the types of sensors with each other to determine the most common value of the number “i”.

According to the above method, regardless of the types of sensors, the same value “i” should be obtained theoretically. If one type of sensors indicate a different result, it is likely that an error occurs, there may be a problem in the sensors of the type and/or the wiring thereof. Thus, this method to specify the sensor (i) by comparing the results is effective not only in accuracy of the measurement, but also in early detection of problems in the sensors. Further, with variety of the sensors 2, various physical conditions may be handled effectively. In addition, when the power supply is turned off at any point throughout the flow shown in FIG. 20, the process is immediately terminated.

Alternatively, through S160 to S164, an inserted length may be calculated in each step, and the thus obtained lengths may be compared with each other to determine a final value. The lengths obtained from each calculation may be aggregated, and the most common length may be determined as the length to be displayed on the monitor.

Alternatively, results from sensors with higher accuracy may be selected. Among the sensors described above, temperature sensors and pressure and vibration sensors indicates results with higher accuracy. Thus, when the specifically precise length is required, results from the temperature sensors or the pressure and vibration sensors may be selected.

Alternatively, results from sensors with higher responsiveness may be selected. Some sensors, such as the temperature sensors or the pressure and vibration sensors may require more time to obtain results than others. Thus, when the length of the flexible tube 4 immediately after the insertion is to be specified, results from the sensors with high responsiveness, such as the optical sensors, may be selected.

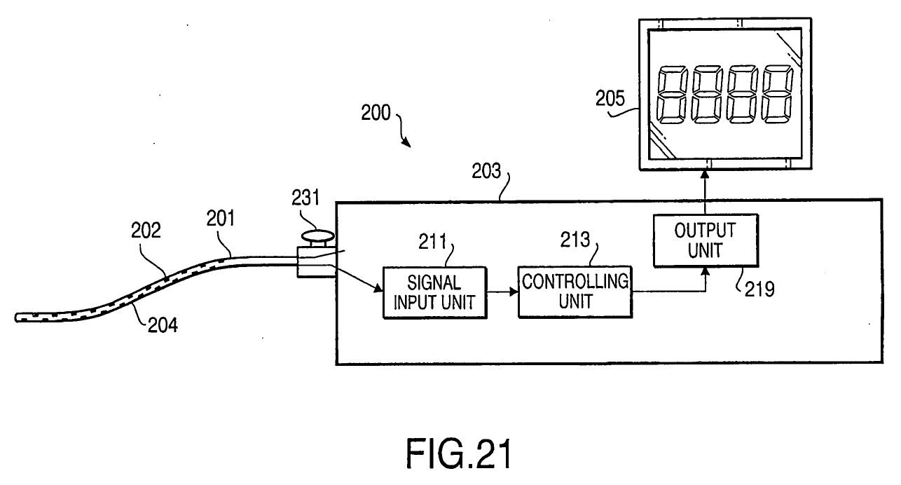

The methods that have been described herein refers to displaying information indicating the inserted length of an electronic endoscope, however, the scope of the present invention is not restrict to only a system with an electronic endoscope. Additionally, an electronic endoscope can be replaced by a fiber-optic endoscope. In that case, a simpler LED display may be used instead of a monitor 5 as shown in FIG. 21.

FIG. 21 is a diagrammatic view of a configuration of an fiber-optic endoscope 200. The fiber-optic endoscope 200 is provided with a fiberscope 201 for observing the affected area, a processor 203 that processes various data, and an LED display 205 as a displaying device to indicate an inserted length of the fiberscope 201.

The fiberscope 201 is provided with a flexible tube 204 that is to be inserted into the body of the patient, an operation unit 231, and a ROM 232 wherein, for example, information on the sensors is stored. In addition, sensors 202 for measuring environmental element surrounding the fiberscope 201 are arranged on the flexible tube 204. For sensors 202, various types of sensors including temperature sensors, optical sensors, pressure sensors, vibration sensors, wetness sensors (i.e. electrodes), and humidity sensors are used, similarly to the sensors 2 that are arranged on the flexible tube 204 described above. The processor 203 is provided with a sensor signal input unit 211 to receive signals obtained from the sensors 202, a controlling unit 213 to process the signals obtained from the sensors 202, an output unit 219 to output the video signals to the LED display 205.

The process implemented by the fiberscope 201 to process the signals obtained from the sensors 202 will be described. Signals obtained by the sensors 202 are inputted to a sensor signal input unit 211, then sent to the controlling unit 213 wherein the signals are processed for the inserted length of the flexible tube 204, similarly to the above-described endoscope system 1. The processed results are transmitted via the output unit 219 to the LED display 205 to indicate the inserted length of the flexible tube 204. In addition, a method to display the information may not be restricted to an LED displaying device, and an insertion length indicator as described in previous embodiments may be used. Furthermore, the insertion length indicator may be overlaid with a pattern diagram of a human body.

The present disclosure relates to the subject matter contained in Japanese Patent Application No. 2004-202136, filed on Jul. 8, 2004, which is expressly incorporated herein by reference in its entirety.

What is claimed is:

1. An endoscope system, comprising:
an endoscope having an insertion section to be inserted into a body cavity;
a plurality of sensors that detects environmental condition thereof, the plurality of sensors being arranged along a longitudinal axis of the insertion section;
a processor that obtains information related to an inserted amount of the insertion section in accordance with detection results of the plurality of sensors; and
a monitor device that displays information related to the inserted amount of the insertion section.

2. The endoscope system according to claim 1,
wherein the processor identifies a sensor located at a boundary between an inserted part and non-inserted part of the insertion section in accordance with the detection results of the plurality of sensors.

3. The endoscope system according to claim 2,
wherein the information related to the inserted amount of the insertion section obtained by the processor is an actual length which is displayed on the monitor as a character string.

4. The endoscope system according to claim 2,
wherein the information related to the inserted amount of the insertion section obtained by the processor is displayed on the monitor as an image indicating an inserted length.

5. The endoscope system according to claim 4,
wherein the image indicating the inserted length includes an insertion length indicator that shows the inserted amount of the insertion section with respect to an entire length of the insertion section.

6. The endoscope system according to claim 5, wherein the insertion length indicator is displayed on the monitor device together with a diagram of a human body.

7. The endoscope system according to claim 5, further comprising a storing system that stores the information related to the inserted length of the insertion section.

8. The endoscope system according to claim 1, wherein the information related to the inserted length is overlaid on an observation image on the monitor device.

9. The endoscope system according to claim 1, wherein the insertion section is defined by a front end and a rear end, wherein the sensors are arranged between the front end and the rear end in a predetermined regularity.

10. The endoscope system according to claim 9, wherein the plurality of the sensors are arranged such that every two adjoining sensors are spaced from each other in an axial direction of the insertion section, while the every two adjoining sensors are located at opposite positions in a circumferential direction of the insertion section.

11. The endoscope system according to claim 9, wherein the inserted length is determined by identifying a sensor which is closest to the front end of the insertion section among other sensors that are arranged on a part of the insertion section that is not inserted into the body.

12. The endoscope system according to claim 9,
wherein the processor determines a boundary between the inserted part and non-inserted part of the insertion section in accordance with a difference between detection results adjoining ones of the plurality of sensors.

13. The endoscope system according to claim 9,
wherein the processor determines a boundary between the inserted part and non-inserted part of the insertion section in accordance with the individual detection results of the plurality of sensors.

14. The endoscope system according to claim 2, wherein the plurality of sensors include at least one of temperature sensors, photo sensors, pressure sensors, vibration sensors, wetness sensors and humidity sensors.

15. The endoscope system according to claim 14, wherein the sensors include the optical sensors.

16. The endoscope system according to claim 15,
wherein the endoscope comprises a flexible tube,
wherein the sensors are arranged on an inner surface of the flexible tube, and
wherein at least portions of the flexible tube facing the optical sensors are formed of optically transmittable materials.

17. The endoscope system according to claim 2,
wherein the plurality of sensors include at least two types sensors, each including the plurality of sensors.

18. The endoscope system according to claim 17, wherein the at least two types of sensors include at least two of temperature sensors, photo sensors, pressure sensors, vibration sensors, wetness sensors and humidity sensors.

19. An endoscope system, comprising:
an endoscope having an insertion section to be inserted into a body cavity for observation of an object therein;
a plurality of sensors that detects environmental condition thereof, the plurality of sensors being arranged along a longitudinal axis of the insertion section;
a monitor device that displays image in accordance with input signals;
an observation image generating system that captures an image of the object and outputs a signal representing the captured image to the monitor device; and
a controller that identifies a boundary of inserted part and non-inserted part of the insertion section in accordance with detection results of the plurality of sensors, the controller generates length information which is transmitted to the monitor device for display.
